(19) United States
(12) Patent Application Publication
(10) Pub. No.: US 2004/0167545 A1
(43) Pub. Date: Aug. 26, 2004

(54) Applicator for surgical clips

(76) Inventors: Kenneth George Sadler, West Sussex (GB); Darren John Giddins, Nottingham (GB)

Correspondence Address: SALTER & MICHAELSON, THE HERITAGE BUILDING, 321 SOUTH MAIN STREET, PROVIDENCE, RI, 029037128

(21) Appl. No.: 10/362,035

(22) PCT Filed: Aug. 17, 2001
(86) PCT No.: PCT/GB01/03736

(30) Foreign Application Priority Data
Aug. 18, 2000 (GB) 0020263.0

(51) Int. Cl. A61B 17/10

(52) U.S. Cl. 606/142

(57) ABSTRACT

An applicator for a sterilization or ligation clip incorporates a life limiter mechanism which prevents usage of the applicator after a present number of clipping operations. The mechanism also preferably includes audible signalling means indicating correct closure of each clip.

### BACKGROUND

1. Technical Field

The present invention relates to applicators for surgical clips and, more particularly to applicators for applying sterilization or ligation clips in surgical procedures.

2. Related Cases

The invention finds particular but not exclusive use in ensuring the performance of an applicator such as described in British patent application No. 9919170.2.

3. Related Art

Applicators of this type are used many times to effect closure of clips. The applicant has discovered that a problem can arise if the applicator becomes worn in use. Presently, the procedure is to examine such applicators at intervals, but there are problems with the procedure especially if, as is often the case, the applicator is used by several surgeons.

In such cases the inspection procedure is often not carried out at the correct interval or the wrong applicator is serviced and this can lead to an applicator being used by a surgeon when it is no longer completely serviceable.

This problem is particularly, but again not exclusively, acute when the applicator, or parts thereof, are made from plastic material as is the case with the applicator in the above mentioned British Patent application.

However, in some circumstances the applicator will be too worn and it will not correctly close the clip even though it may appear to the surgeon to have correctly operated. This is extremely dangerous since if the surgeon considers that the clip has been correctly applied it will be assumed that the operation has been successful. The clip may remain in position for a period but may then become dislodged leading to failure which in the case of a sterilization clip could lead to pregnancy and in the case of a ligation clip could lead to loss of blood and possible complications resulting therefrom.

Presently, the procedure is to examine such applicators at intervals, however, incorrect records of servicing at the hospital/clinic will result in applicators being used past the recommended service period or number of uses. Protracted use of reusable instruments past the recommended service date or designated number of usages may produce worn parts as well as flexion where force is transmitted through the clip closure area of the instrument; this can have the effect of closing clips that are not sufficiently closed and problems may result that are not detectable by the surgeon. This is extremely dangerous.

U.S. Pat. No. 5,478,003 discloses a pneumatically powered surgical fastening device including a life limiter and including means for indexing the number of operations performed by the application preventing resetting of the life limiter means.

It is the object of the present invention to provide a means to protect the performance of a reusable device within a performance envelope (identified by the manufacturer) that is considered to be acceptable by introducing Usage limiting means in order to negate the need for servicing to the end user. It is the object that this invention will allow products with life limited functions to be sent to end-users who are geographically remote from the nearest authorised service department.

It is also the object of this invention to provide means to detect the force required to close the clips such that a life is not counted unless the correct force is applied. A problem will therefore be detected if a count is not detected.

It is an object of the present invention to provide a life limiter means in the applicator which restricts the usage of the applicator. Most preferably the means provided is arranged so that it cannot be tampered with once the applicator has been assembled.

Thus in a specific example if the life limiter means is set to, for example, 500 operations, the surgeon will not be able to use the applicator after this number of clip applications has been performed.

### SUMMARY

Preferably, the applicator will have a readout means enabling the surgeon to observe the number of operations remaining. This may be, for example, through a window in the handle. The more clips used will reduce the number of operations available. This will enable the surgeon to know when the applicator is nearing the end of its design life and to ensure that a new applicator is available.

In a preferred embodiment the applicator life limiter should be able to be set to a defined number of clip applications, this number being variable to suit different usages of the applicator, for example, when different clips—sterilization or ligation are to be closed by the applicator.

The invention preferably provides a capsule assembly to hermetically seal the usage limiting assembly from contamination within the intricate mechanism. In a preferred embodiment the applicator life limiter should be able to be set to a defined number of clip applications. Different lives will be achieved by altering the gear train on the cam and ratchet wheels.

For different instrument requirements, different Life limiter capsules representing different lives can be assembled. The capsule assembly provides a means for testing the mechanism prior to assembly within the main body of the instrument such that once sealed it cannot be tampered with. This invention allows one company to be responsible for the manufacture and testing of the life limiter parts irrespective of when or where the handle assembly of an instrument is assembled.

The present invention therefore provides an applicator suitable for sterilization or ligation clips as claimed in claim 1.

Preferably, the life limiter includes a disengagement means which permanently disengages an actuation mechanism in the applicator after said predetermined limit.

Preferably, said limiter means includes usual indicator means to provide an indication of the instantaneous number of applications which has already been performed by the applicator.

Preferably the disengagement means comprises sprag means and flange means attached to a count wheel said flange means comprising a generally circular main portion and a cut away eccentric portion at a predetermined position on said flange means said count wheel being incrementally turned by a ratchet wheel and in which said sprag means normally rests on said generally circular main portion until said eccentric position is rotated to be in contact with said sprag means and in which when said sprag means contacts said eccentric portion of said flange means said sprag means disengages from a first position to a second position, at which second position said life limiter becomes non-operative.

Preferably said sprag means contacts a disc spring which resists the force necessary to allow forward movement of a pawl life limiter tooth mounted on said ratchet wheel.

### BRIEF DESCRIPTION OF THE DRAWINGS

Embodiments of the present invention will now be described by way of example with reference to the accompanying drawings in which:

FIG. 1 shows in partial cross section an applicator illustrating the life limiter of the present application;

FIG. 2 shows the position of a sealed enclosure life limiter unit within the handle of the applicator;

FIG. 3 diagrammatically shows the sealed enclosure life limiter unit in a perspective view;

FIG. 4 diagrammatically shows the front side portion of the sealed enclosure life limiter;

FIG. 5 shows schematically a perspective view of the sealed enclosure life limiter of FIGS. 2 to 4 illustrating the position of the internal components;

FIG. 6 shows the operating lever seal of the sealed enclosure in greater detail;

FIG. 7 shows the rear casing of the sealed enclosure life limiter without internal components;

FIG. 8 shows the life limiter unit and primary trigger in vertical cross section;

FIG. 9 shows the life limiter unit with the primary trigger of the applicator on a neutral position;

FIG. 10 shows the unit of FIG. 11 with the trigger in a closed position;

FIG. 11 shows the life limiter of FIGS. 11 and 12 in greater detail;

FIG. 12 shows the seal capsule life limiter with the primary trigger in a neutral position;

FIGS. 13 and 14 show front and rear perspective views of the internal components of the sealed life limiter unit;

FIGS. 15 to 18 illustrate various positions of the sprag and pawl mechanism;

FIGS. 19 to 23 illustrate in greater detail the operation of the pawl and sprag mechanisms; and

FIGS. 24 and 25 illustrate positions of the spring in the rest and operated positions.

### DETAILED DESCRIPTION OF THE PREFERRED EMBODIMENTS

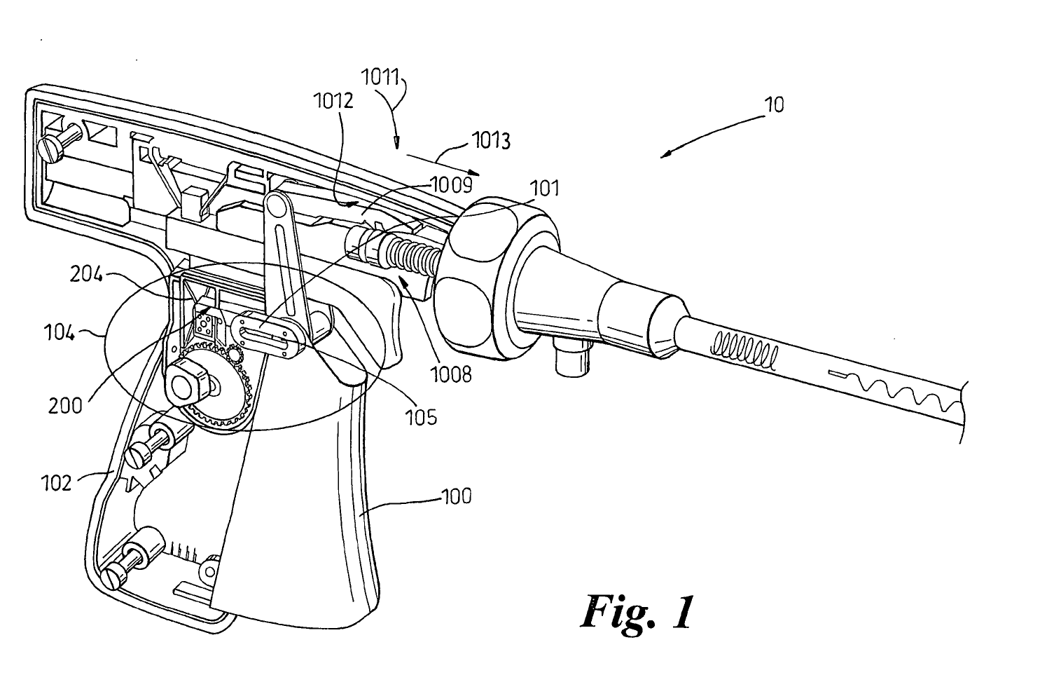

The operation of the life limiter will now be described initially in outline with reference to FIG. 1 and then in greater detail with reference to the remaining figures. With reference to FIG. 1 of the drawings, an applicator 10 is shown for applying clips which could be a sterilization or ligation clips and operation of a main trigger 100 transfers motion to the further operating mechanism by means of a lever action. Rotation takes place about the trigger pivot point 105 and reaction forces generated at this point allow the trigger to apply the required force to the further components of the mechanism. The reaction forces are taken up in the pivot bearings 101 which are either formed integrally or assembled in each side of the pistol grip 102. In the selected mechanism indicated by the outline circle 104, the pivot bearings are split and the compression half is supported by a collapsing link 200. As long as the link is maintained in its rigid compression state, the trigger action is maintained. When the support provided to the trigger pivot by the link components 200 is removed, the trigger becomes free and cannot drive the further components of the clipping mechanism.

When designing the collapsing link, the opportunity has been taken to incorporate a further feature into the mechanism. A requirement previously established was to incorporate some audible confirmation that the clipping operation had been completed. This has been achieved by means of a disc spring 204 that is activated by the motion of the main trigger 100, signaling the end travel of the mechanism by an audible click. The mechanism now proposed to count clipping operations has been elaborated to count only clipping operations that have achieved the level of force required to deform a clip. The resulting mechanism incorporates provision to monitor the force and trigger stroke required to achieve complete clipping and to give the audible signal and register a count only when the appropriate conditions have been achieved. The mechanism emits one distinctive click and registers a count when the presence of a clip in the applicator jaw has caused sufficient resistance to motion to deflect the disc spring 204 incorporated into the Life Limiter mechanism. A further, different, click is emitted when the mechanism reaches full travel. By registration of and interpretation of these audible signals, the surgeon may be assured that a clip has been deformed and fully closed.

The mechanism incorporates a simple counter based upon a ratchet mechanism plus a gear reduction, required to achieve the desired maximum count. The final drive wheel incorporates a cam that supports the components of the collapsing link in their extended position until rotation of the cam has reached a defined point. At this point, the cam circumference is cut away and the support to the link mechanism is removed, disabling the trigger lever mechanism.

The mechanism consists of the following components.

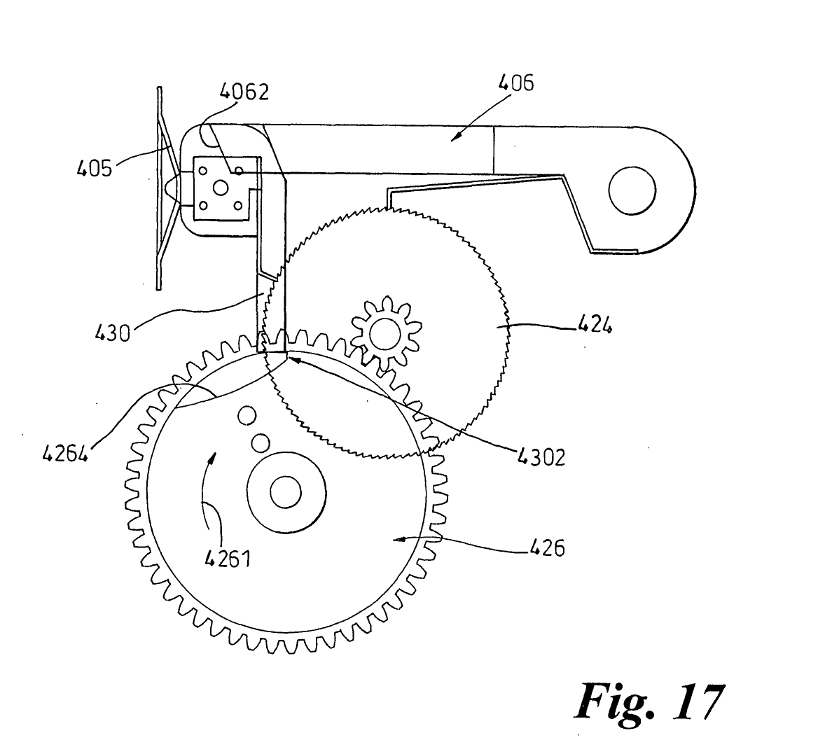

Pivot Support and Pawl 406 (FIG. 17) which has an inclined face 4062 at a left hand end which is shown in detail in the modified embodiment in FIG. 17.

Sprag Component 430 which co-operates with the pawl component 406 and is shown in further detail in FIG. 17.

Ratchet Wheel

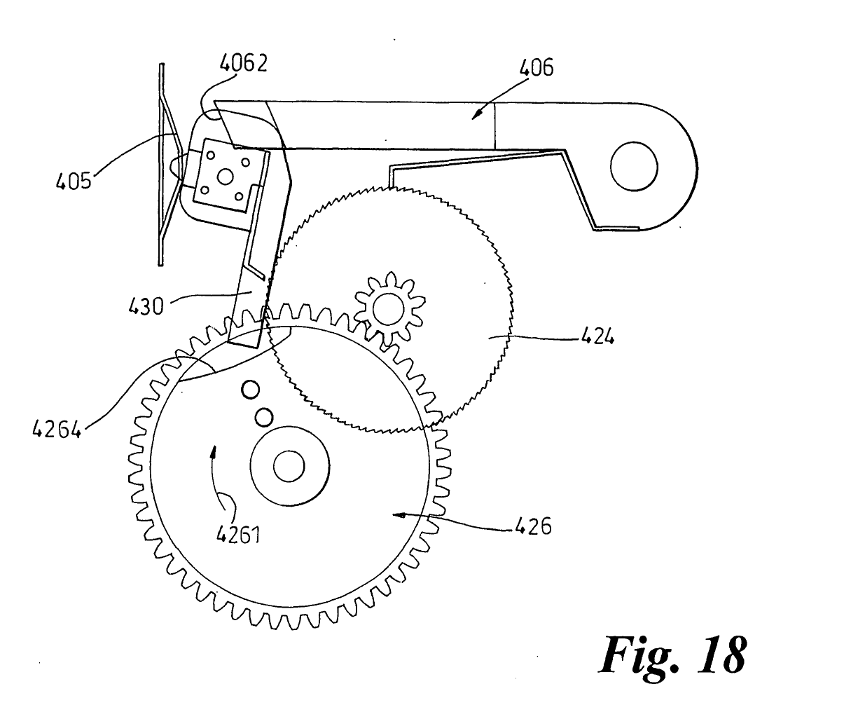

The main pawl engages with the teeth of a ratchet wheel 424 (see FIG. 18) in such a way that lateral movement of the “pawl” component is transmitted to the wheel, resulting in incremental rotation if the “pawl” component moves back and forth through a sufficient distance. A pinion formed on the ratchet wheel transmits rotary motion further to a cam wheel 426.

Cam Wheel

A circular, concentric edge cam 4262 formed on the wheel 426 provides support to the cranked leg of the “sprag” component up to a point of rotation where the cam periphery is cut away.

Disc Spring

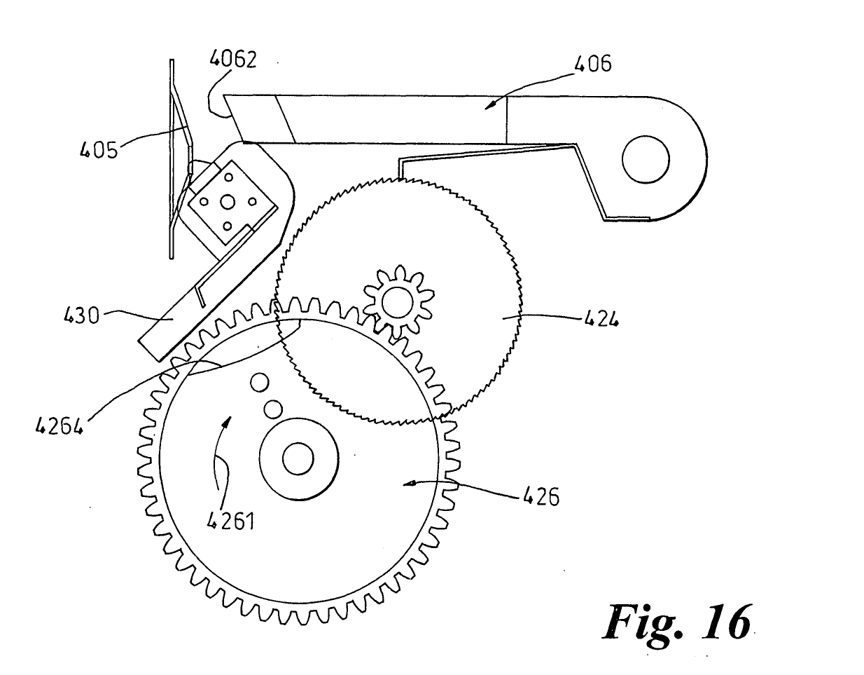

The disc spring 204 (or 405—FIG. 16) may for example be produced by plastically deforming a circular disc in such a way that a subsequent loading tending to flatten the disc results in a strongly non-linear force/deflection characteristic. When loaded axially, little or no deflection of the disc takes place up to a threshold force. Beyond this force, deflection proceeds at a much greater rate up to the point where the disc is substantially flat. At this point the restoring force is positive but may be less than the threshold force. In deflecting under forces above the threshold value, the spring accelerates greatly and causes an audible “click” at the limit of travel.

In the assembled state, the combination of the disk spring, the “sprag” and the “pawl” components retain the trigger pivot in the defined location with a preload. The trigger may then operate on the mechanism to produce clipping. If no clip is loaded in the applicator jaw or the force referred back to the trigger pivot does not reach the spring threshold, then no deflection of the spring takes place, no click is produced and no motion of the ratchet wheel results. If a clip in the jaw is correctly deformed, then force above the threshold value causes the disc spring to deflect fully, a click is produced and the ratchet wheel is moved on one tooth.

After a number of valid clip closing operations, determined by the relative angular position of the cam wheel when initially assembled, the trailing edge of the leg of the “sprag” component reaches the point where the cam profile is cut away. At completion of the next valid clip closing operation, the spring deflects, the compression strut and the leg moves to the left and the leg falls into the cavity of the cam. This motion is initiated by the compression force in the strut, via the inclined faces between the two components of the strut. The collapse of the mechanism is irreversible, without complete disassembly of the actuator body. The body would be sealed against tampering.

An indication of the number of valid clip closing operations that have been carried out and of the remaining life of the applicator can be obtained from the rotation of the cam wheel. By incorporating a suitable further cam profile, a moveable indicator can be driven and this can be arranged with an “expanded scale” giving a more precise indication of the remaining number of operations in, say, the last ten. This may be achieved by tailoring the profile of the cam.

The number of allowable operations before collapse of the mechanism can be selected at assembly, up to the maximum design number, by installing the cam wheel with increasing rotation back from the collapse point.

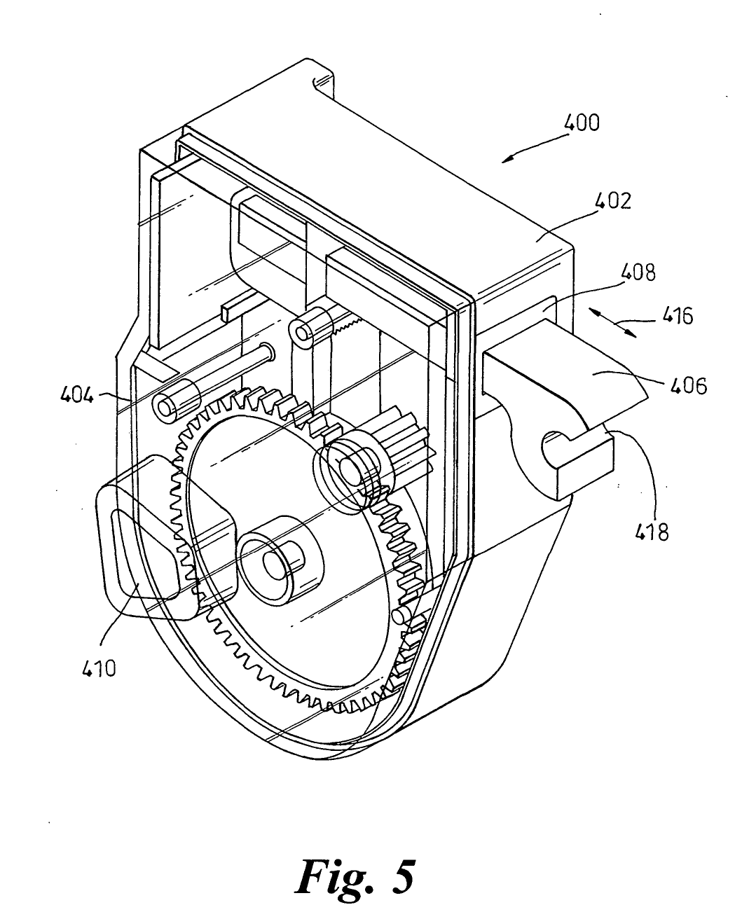

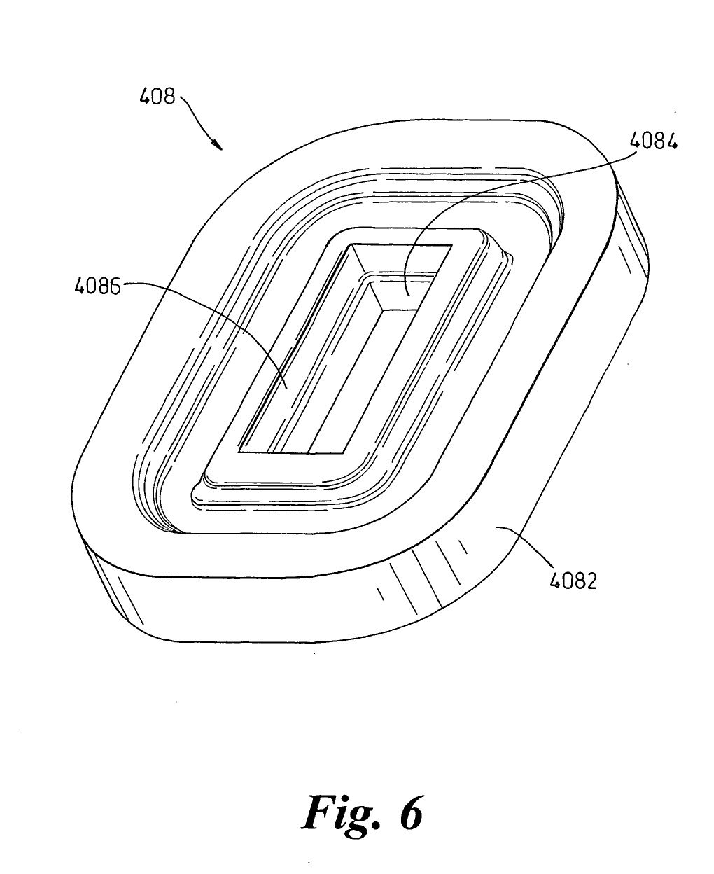

The motion of the cam wheel, and hence the number of elapsed clip counts can most simply be observed through a window 410 (see FIG. 5) in the side plate of the applicator handle. A graphical display is provided on the face of the cam wheel, at the radial window position and extending around the face of the wheel. As the cam wheel rotates, this rotation can be observed and measured by means of graduations on the display area. These graduations may take the form of colored sectors, covering relatively large numbers of individual clip counts and/or individual graduations representing single clip counts. Practical observation of graduations representing single clip counts will require magnification to be included in the viewing window. This should take the form of a cylindrical lens oriented with the cylinder axis along a radius of the cam wheel. The actual count number may be incorporated at suitable intervals but individual identification of every count throughout the range will not be practicable, on the basis of practicable space and magnification.

Further, more complex indicator mechanisms offer the possibility of providing indication at other locations on the applicator, together with increased “scale length” and variable sensitivity over the complete range. Using these techniques, the display of individual clip counts in, say, the last 50-100 becomes feasible. The initial stages of the range would present information either in the form of colored sections, with the possible addition of actual counts at cardinal intervals. By these means, the user can see at a glance whether the remaining operating life of the applicator is large and hence not of detailed interest or within the “red” zone where a small number of operations remains—in this case the actual operations are easily read off and checked down to zero and disablement of the device.

With reference to the life limiter apparatus as shown in FIG. 1 in order to enable the life limiter to operate over a large number of operations the gearing and tooth size required in a handle suitable for operation by hand means that the size of the components must be relatively small.

Since the applicator is used in a medical environment the applicator will require to be sterilized between operations which requires robust operation of the life limiter which is in effect at odds with the necessity for relatively small and accurately machined components.

The inventive solution embodied in the further figures is to provide the life limiter as a sealed unit within the handle. By providing the life limiter as a sealed unit it is possible to include accurately machined miniature components which will not be contaminated within the applicator and therefore will not require to be sterilized.

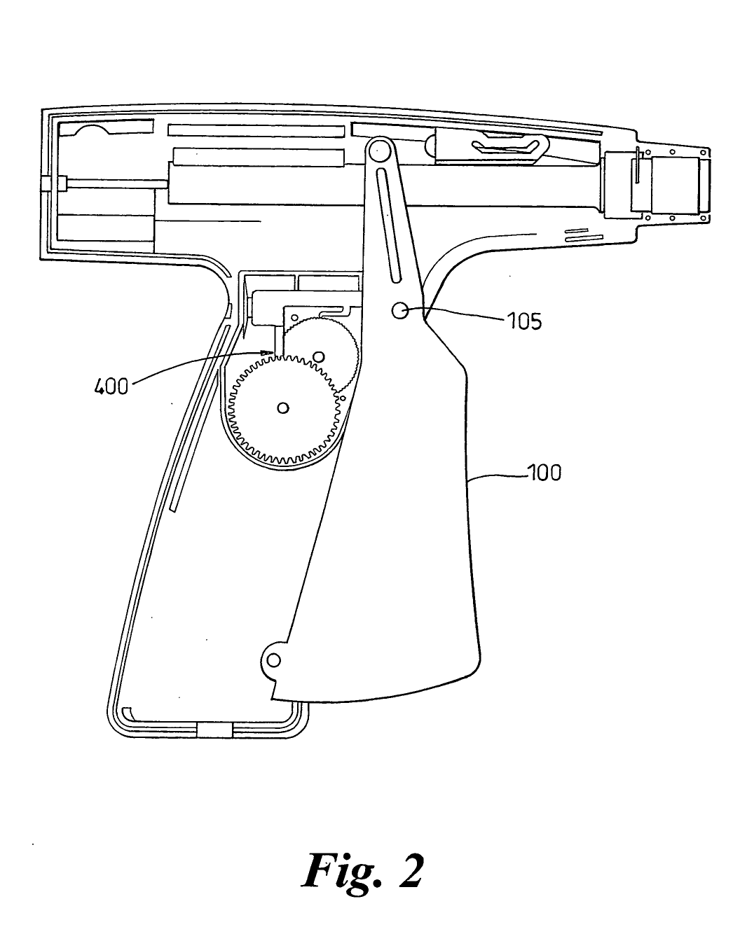

With reference now to FIG. 2 in the practical embodiment the position of the sealed unit 400 is shown relative to the applicator handle 100.

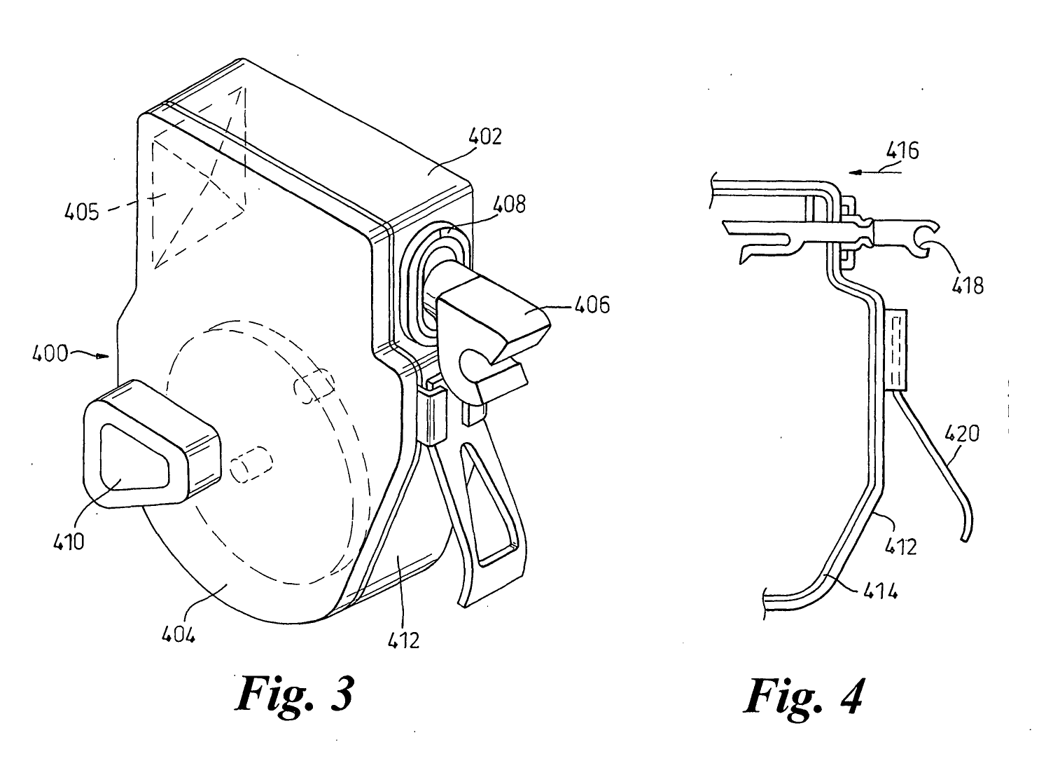

The sealed unit 400 is shown schematically in FIGS. 3 and 4. Details of unit 400 are shown in the further figures.

FIG. 3 shows unit 400 in a perspective side view illustrating the principle features. The unit 400 has a back box section 402 within which the mechanism is assembled as described hereinafter. A front cover section 401 is sealingly affixed to the rear section in the completed unit assembly. The life limiter unit 400 is activated by control lever 406 which enters unit 400 by means of a seal 408 which ensures that the inner workings of unit 400 are sealed against ingress of any contamination during operation of the applicator.

The unit 400 includes in its front cover 404 a viewing window 410 which is preferably magnified to enable easy viewing of the number of operations already performed by the applicator.

With reference to FIG. 4 the front side portion 412 of life limiter unit 400 is shown including a seal 414 which seals the front and back covers.

The seal 408 provides means to allow operating lever 406 to move in a direction indicated by arrow 416. Lever 406 includes engagement means 418 which engages with main handle 100. A return spring 420 is attached to unit 400 and provides return spring force to handle 100.

With reference to FIG. 5 unit 400 is shown schematically in a perspective view to illustrate the position of the internal components with front cover 404 being a clear plastic allowing a view of the internal components to be described in greater detail hereinafter.

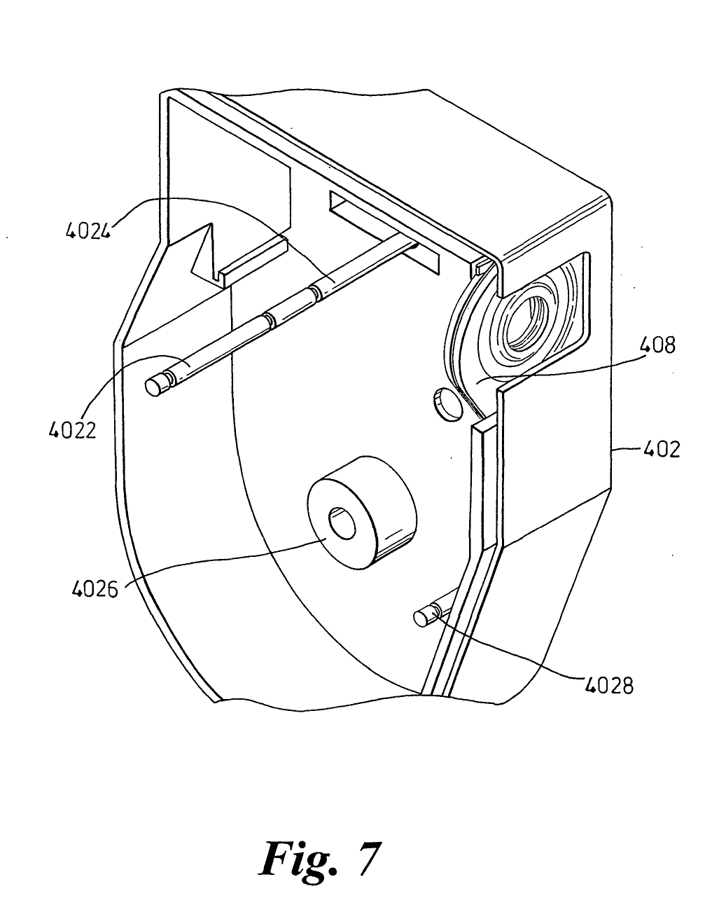

With reference to FIG. 6 seal 408 is shown in greater detail. The seal comprises an outer portion 4082 which seals with the rear portion of cover 402 and a inner whole portion 4084 which seals control lever 406. The seal further preferably comprises flexible corrugated portion 4086 which enables lever 406 to move in the direction of arrow 416 (see FIG. 6) whilst the central inner whole portion 4084 seals onto lever 406 without movement of the lever through the seal. The seal thereby provides a seal which is moisture and watertight and therefore prevents ingress of any material which may be present in operating theaters where the applicator is used. The seal 408 could alternately be of a stretchable membrane type being permanently fixed to control lever 406 ensuring an absolute seal around lever 406 the membrane moving or x stretching with movement of lever 406.

With reference to FIG. 7 the rear casing 402 is shown without any internal components axle bearing numbers 4022, 4024, 4026 and 4028 which carry the internal components are down. The seal 408 is shown with an alternative circular central aperture, which could accommodate a cylindrical operating lever 406.

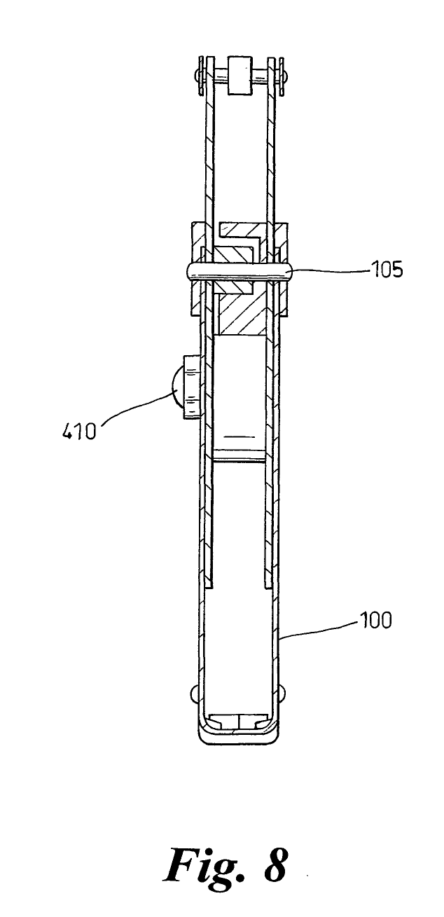

FIG. 8 shows the life limiter unit in a vertical cross section illustrating the relative position of the components.

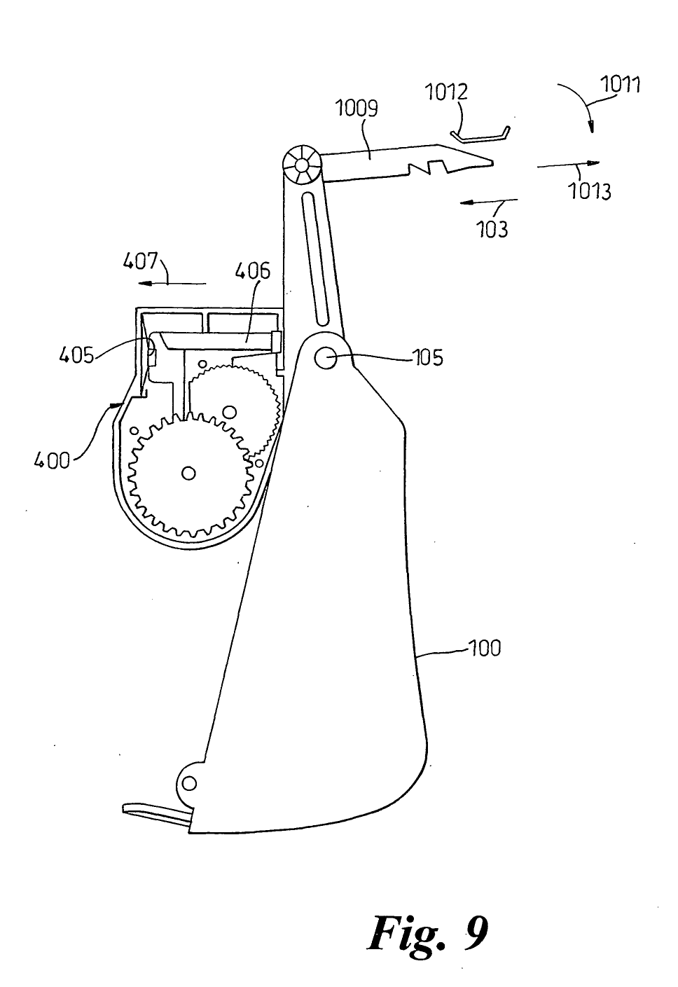

FIG. 9 shows the life limiter unit 400 and primary trigger 100 with the primary trigger 100 in a neutral, non operated position. The primary trigger 100 can be closed when there is no clip in the tray of the applicator as described in co pending application No. 9919072.2 but when no clip is present in the tray then the life limiter is not activated because the primary trigger will not provide sufficient force to move the lever 406 sufficiently to active the pawl in the life limiter unit 400 as described hereinafter. This is because there is not sufficient resistive force on the lever 101 in the direction of arrow 103 to move lever 406 sufficiently in direction of arrow 407.

Movement of trigger 100 will initially move drive lever 1009 in the direction of arrow 1011 to engage the applicator drive system 1008 (FIG. 1) and then in the direction of arrow 1013.

However, when there is a clip present in the open end tray section of the applicator, in a position to be inserted in the correct position to occlude a fallopian tube, there is a reverse force exerted in the direction of arrow 103 caused by the force necessary to permanently deform and close the clip. This reverse force causes the primary trigger 100 to move the operating lever 406 backwards in the direction of arrow 407 and only when the reverse force exceeds a predetermined limit set by the force required to completely deform and close the clip will the lever 406 be moved against spring 405 (FIG. 6) a sufficient distance to operate the unit 400 to record a count. Thus only when there is a clip in the tray can the trigger 100 move lever 406 backwards along its bearing surfaces to activate the count on the life limiter. This rearward displacement compresses spring 405 and produces an audible click

Operation of the life limiter is therefore not possible when the applicator is not being correctly used.

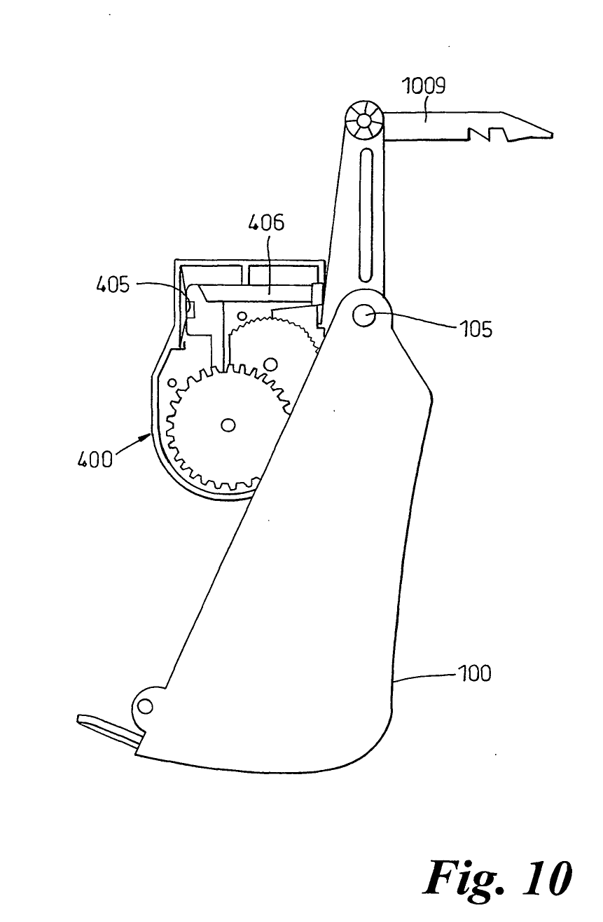

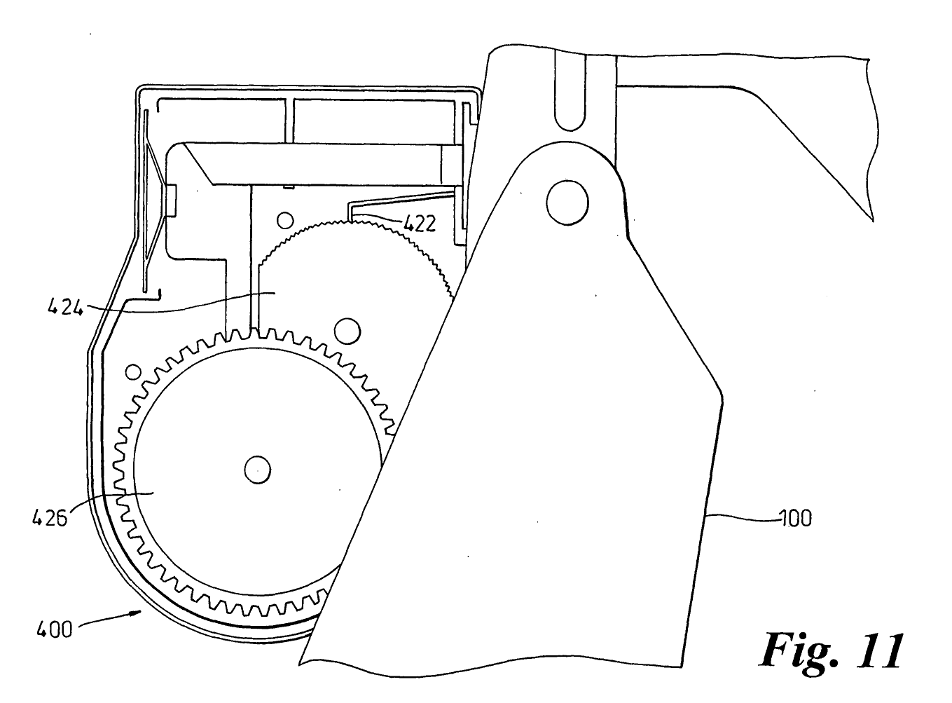

FIG. 10 shows the unit 400 with the primary trigger 100 in the closed or operated position.

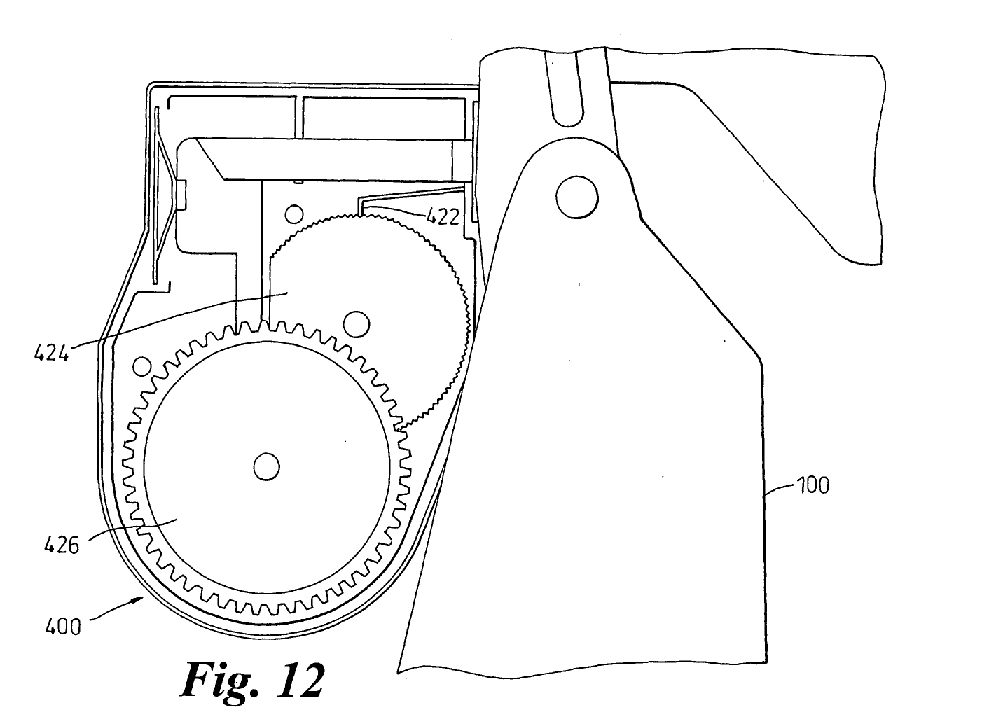

FIG. 11 shows the life limiter unit 400 with the primary trigger in a closed position and illustrating the position of pawl 422 engaging with the ratchet wheel 424 which in turn drives count wheel 426.

FIG. 12 shows the life limiter sealed capsule unit 400 with the primary trigger 100 in the neutral position.

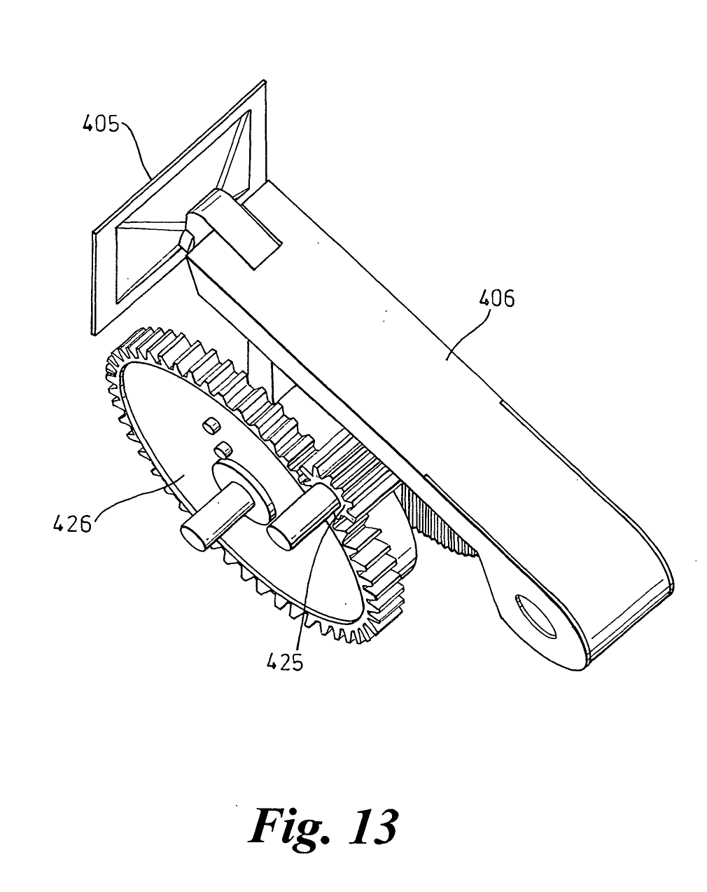

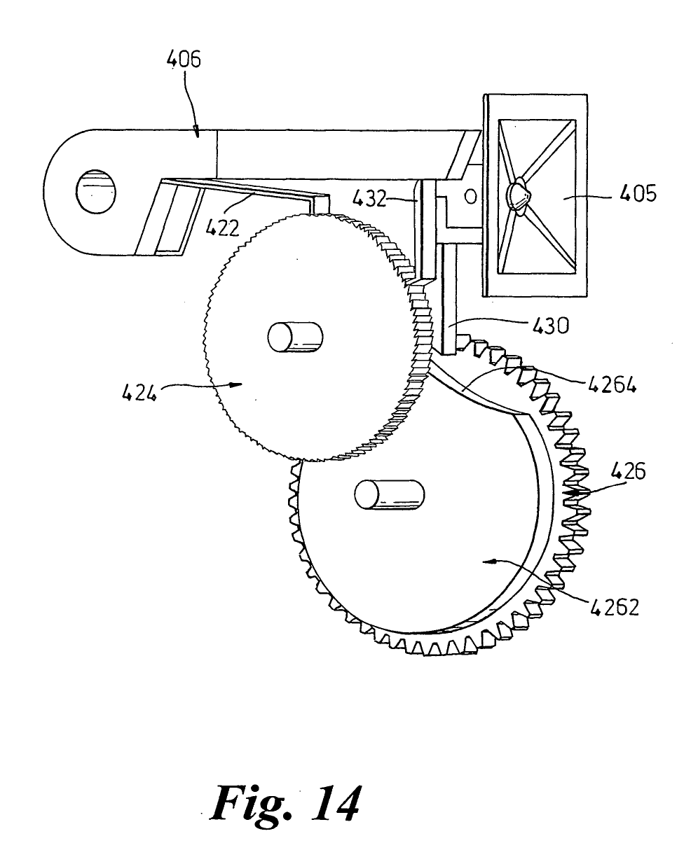

FIGS. 13 and 16 show the internal components of the sealed capsule life limiter unit 400 showing the sprag on the verge of disengagement. The FIG. 13 shows a front perspective view and FIG. 14 a rear perspective view.

These views show in greater detail one possible design of the spring 405 which (see FIG. 3) is secured into the rear face of the unit 400. The spring 405 is preferably made of a spring steel plate structure divided into four segments which provides the desired collapsible movement ensuring that the unit operates to count only when sufficient force is applied to the spring by the lever 406. This collapsible movement provides the desired click sound indicating correct application of a clip.

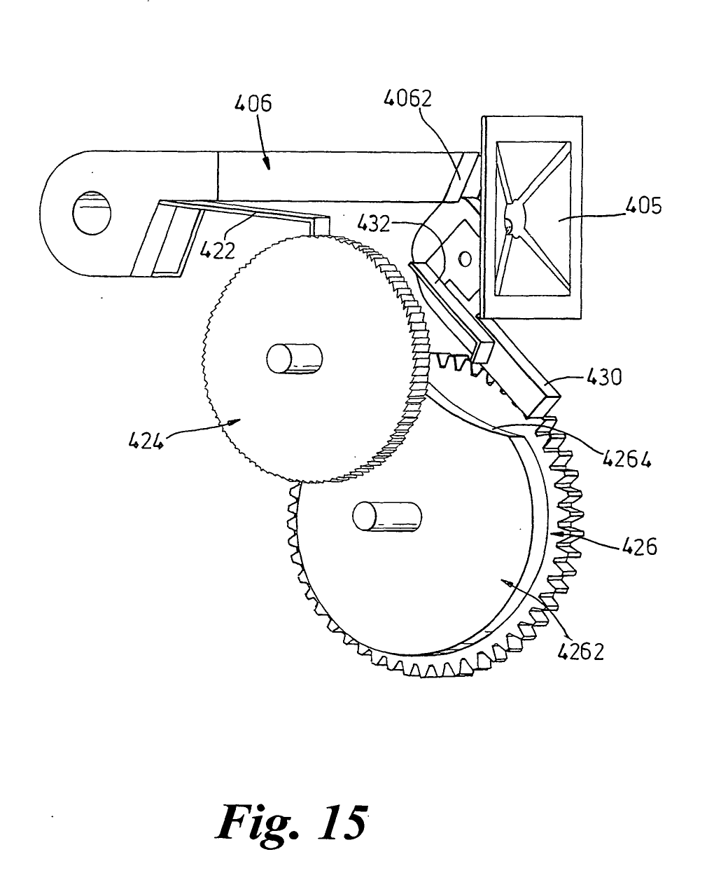

With particular reference to FIG. 14 the rear of count wheel 426 is shown. The count wheel 426 is moved as shown in FIG. 15 by intermediate gear 425 which rotates the count wheel 426 in a desired ratio which can be selected in accordance with the desired number of operations which the life limiter allows. Additionally the initial or starting positions of the gear wheels 424 and 426 can be adjusted during manufacture to provide a desired count.

The life limiter expires or fails when sprag 430 moves off the circular flange surface 4262 of wheel 426 and on to the eccentric curve portion 4264. This occurs when count wheel 426 reaches the predetermined position as illustrated in FIG. 14. A further pawl lever 432 ensures correct movement of wheel 424 ensuring no return of wheel 424.

In FIG. 15 the sprag 430 is shown fully disengaged. As can clearly be seen the end portion 4062 of lever 406 may be clearly seen to be separated from the spring 405. Thus when pressure is applied to the primary trigger 100 there is no resistance from the lever 406 and consequently no substantial pressure can be applied to any clip in the operational tray of the applicator. The applicator will therefore cease to function and the life limiter will have performed its function.

FIG. 16 is a diagrammatic side view again illustrating the position of the sprag as it fails by moving downwards guided by eccentric cam 4264.

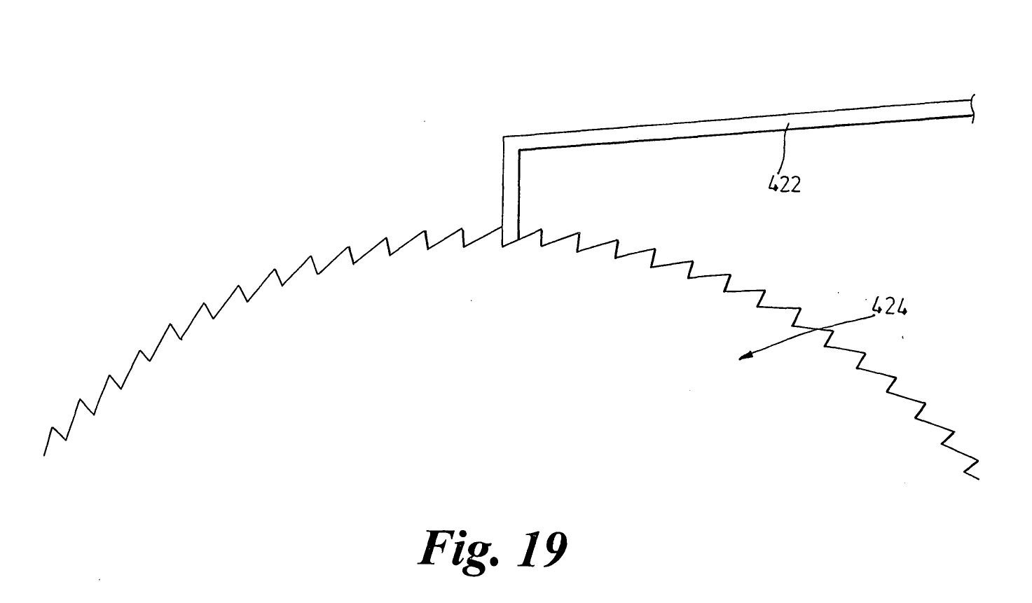

The movement of sprag 430 on disengagement is further illustrated in FIGS. 17 and 18. In FIG. 19 the edge corner 4302 is shown just resting on the edge of the eccentric cam 4264 showing that further rotational movement of wheel 426 in the direction of arrow 4261 as illustrated in FIG. 18.

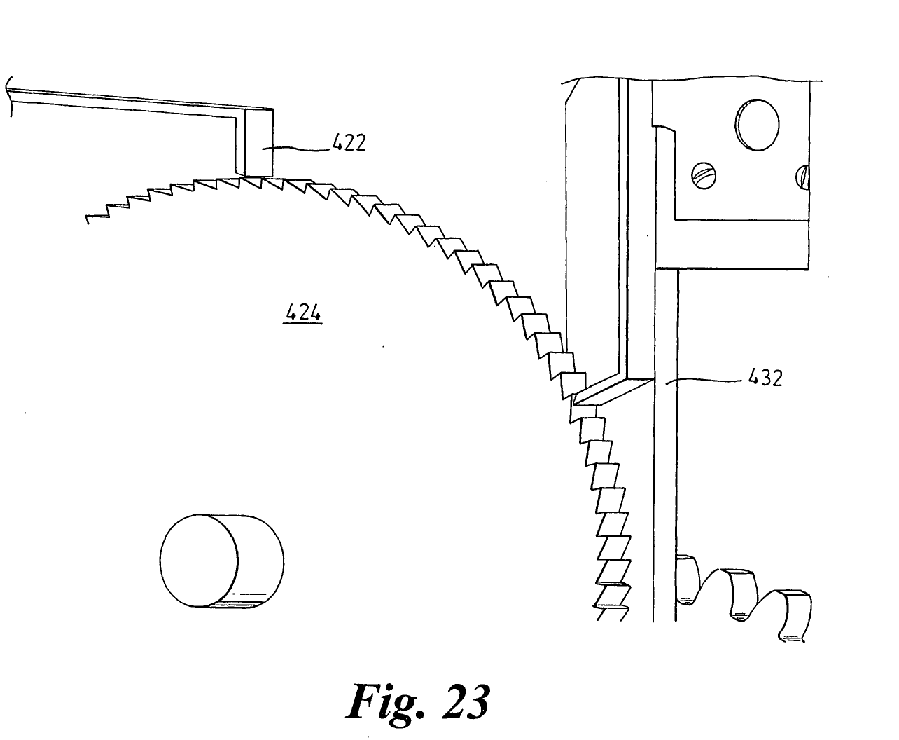

FIGS. 19 to 23 illustrate the operation of the pawl and sprag springs engaging with the teeth on ratchet wheel 424.

FIG. 19 shows the pawl metal jacket 422 engaging with the ratchet wheel 424.

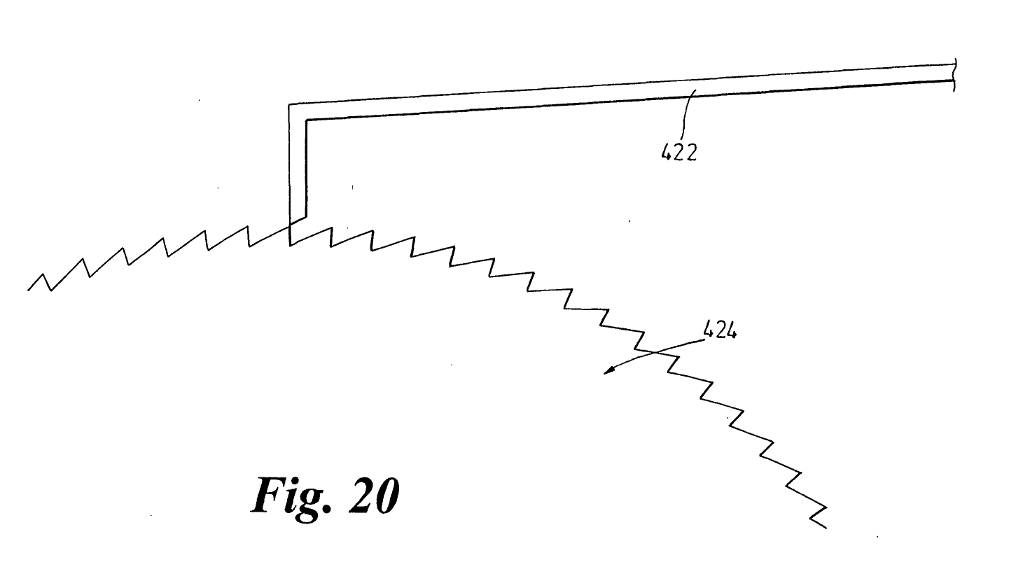

FIG. 20 shows the pawl metal jacket 422 moving over the incremental teeth in the ratchet wheel 424.

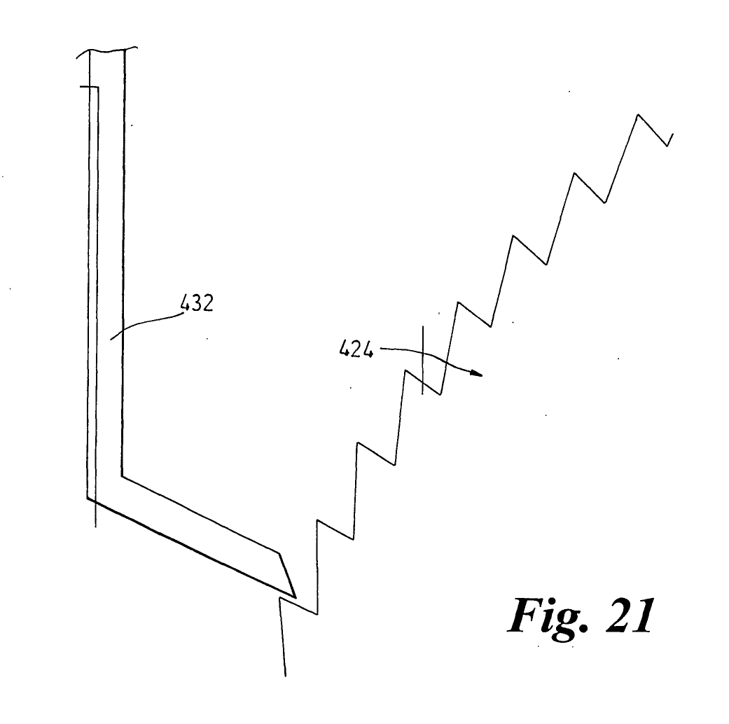

FIG. 21 shows the sprag spring 432 moving over the ratchet wheel incremental teeth,

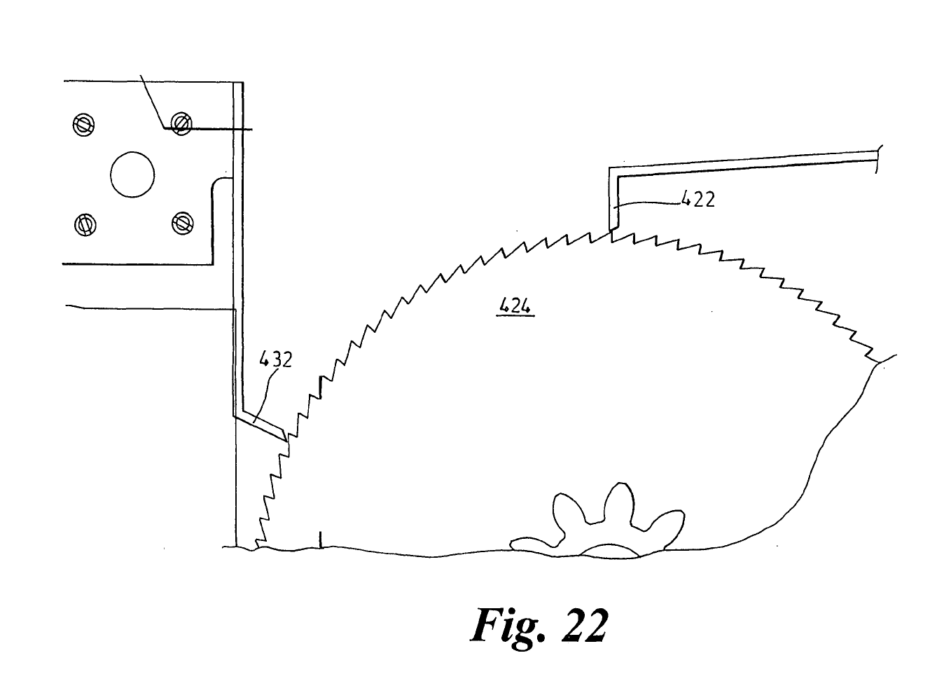

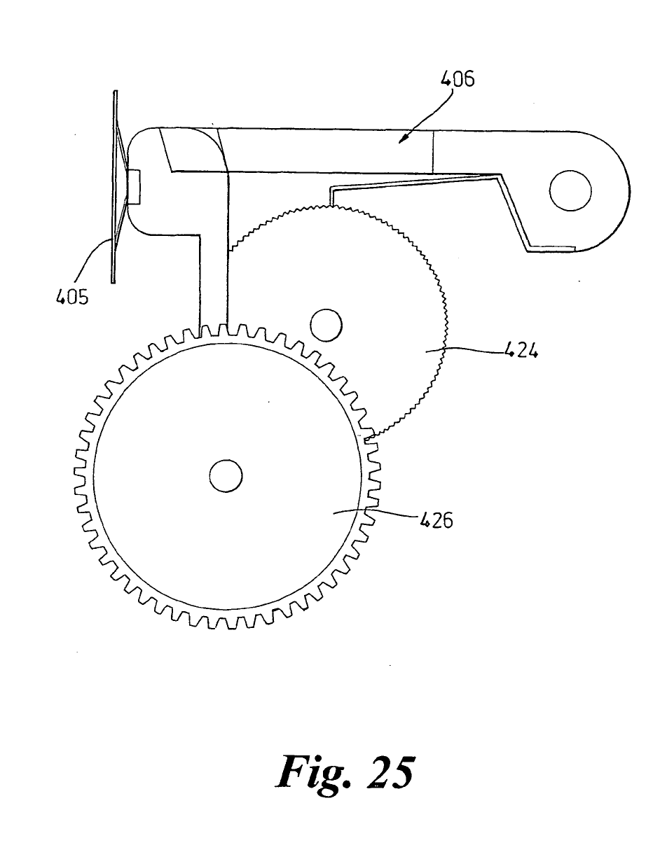

FIG. 22 shows diagrammatically both the sprag spring 432 and pawl spring moving over the teeth in the rachet wheel 424 and FIG. 25 shows a preferred embodiment of the arrangement of FIG. 22.

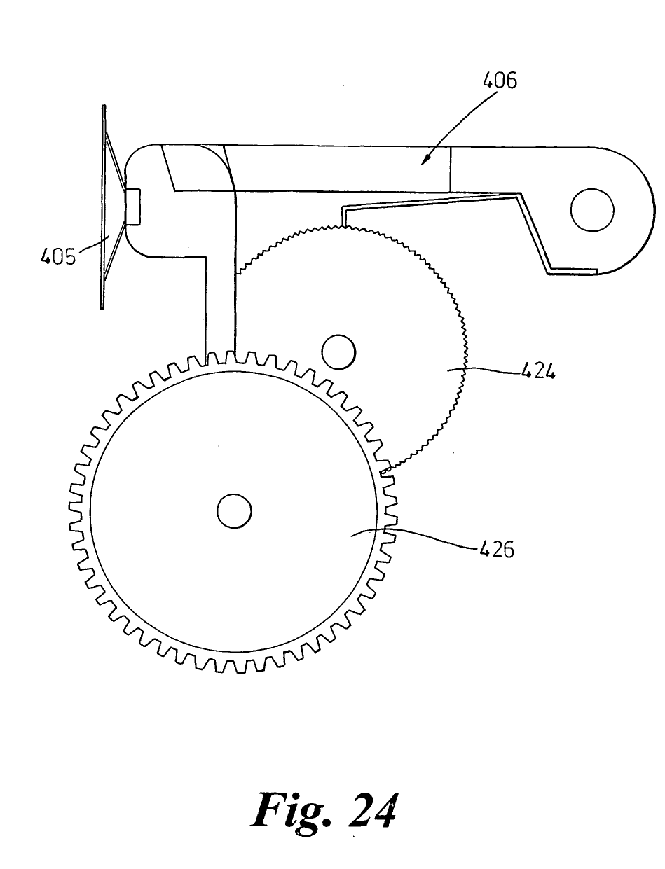

FIG. 24 schematically shows the pawl spring engaged with the ratchet wheel 424 and illustrating the spring 405 in its rest position.

FIG. 25 illustrates the pawl spring moving over the ratchet wheel 424 with the spring 405 being compressed. As described above when the spring 405 is sufficiently compressed it will emit an audible click to indicate completion of the clip closure The compression force of the spring 405 may be selected according to the design criteria which reflects the force required to effect correct closure of a clip in the applicator. Likewise the distance moved by the spring 405 can be designed to suit different applicator designs.

1. An applicator for sterilization or ligation clips, said applicator being provided with life limiter means which is operative to limit the number of clip applications performable by said applicator to a predetermined limit.

2. An applicator as claimed in claim 1 in which the life limiter includes a disengagement means which permanently disengages an actuation mechanism in the applicator after said predetermined limit.

3. An applicator as claimed in claim 1 or claim 2 in which said limiter means includes visual indicator means to provide an indication of the instantaneous number of applications which has already been performed by the applicator.

4. An applicator as claimed in any one of claims 1 to 3, including audible confirmation means connected between an operating handle of said applicator and said life limiter means, said audible confirmation means providing an audible click signal indicating at least one predetermined level of resistance to the movement of the handle.

5. An applicator as claimed in claim 4 in which said audible confirmation means comprises a disc spring.

6. An applicator as claimed in claim 4 in which a first predetermined level of resistance is determined to indicate a first pressure on said disc spring indicating a first relatively low pressure resistance of the movement of the handle.

7. An applicator as claimed in claim 5 in which a second predetermined level of resistance is determined to indicate a second pressure on said disc spring indicating a second relatively high level of resistance of the movement of the handle.

8. An applicator as claimed in any one of claims 2 to 7 in which the disengagement means comprises sprag means and flange means attached to a count wheel said flange means comprising a generally circular main portion and a cut away eccentric portion at a predetermined position on said flange means said count wheel being incrementally turned by a ratchet wheel and in which said sprag means normally rests on said generally circular main portion until said eccentric portion is rotated to be in contact with said sprag means and in which when said sprag means contacts said eccentric portion of said flange means said sprag means disengages from a first position to a second position at which second position said life limiter becomes non-operative.

9. An applicator as claimed in claim 8 in which initial said sprag means contacts a disc spring which resists the force necessary to allow forward movement of a pawl life limiter tooth mounted on and ratchet wheel.

10. An applicator as claimed in any one of claims 1 to 8 in which said life limiter means is contained within a sealed enclosure.

11. A life limiter unit comprising a sealed enclosure unit as claimed in claim 9 suitable for a sterilisation clip applicator.
